(19) United States
(12) Patent Application Publication
(10) Pub. No.: US 2023/0181320 A1
(43) Pub. Date: Jun. 15, 2023

(54) EXPANDABLE ANNULOPLASTY RINGS

(71) Applicant: EDWARDS LIFESCIENCES CORPORATION, Irvine, CA (US)

(72) Inventors: Louis A. Campbell, Santa Ana, CA (US); Rodolfo Rodriguez, San Luis Obispo, CA (US); Da-Yu Chang, Irvine, CA (US); Gregory Bak-Boychuk, San Clemente, CA (US); Brian S. Conklin, Orange, CA (US); Amy E. Munnelly, Irvine, CA (US)

(21) Appl. No.: 18/167,720

(22) Filed: Feb. 10, 2023

Related U.S. Application Data
(63) Continuation of application No. PCT/US21/71152, filed on Aug. 10, 2021.
(60) Provisional application No. 63/064,287, filed on Aug. 11, 2020.

(51) Int. Cl. A61F 2/24

(52) U.S. Cl. CPC A61F 2/2448; A61F 2250/0029; A61F 2250/0036

(57) ABSTRACT

A mitral repair annuloplasty ring that will accommodate implantation of a transcatheter valve therein for a valve-in-ring reoperation. The ring has a semi-rigid core with features that allow the ring to better conform to the cylindrical geometry of a transcatheter valve when implanted. The inner core defines a continuous peripheral D-shape with a substantially straight anterior side diametrically across from a more rounded posterior side with arcuate lateral sides therebetween. Segments of the ring core are subject to differing rates of bending due to variable radial thicknesses or radial slits opening to both inner and outer edges around the core periphery. One or more expansion joints may also be used to create the more circular final expanded shape. A plastically expandable sealing sleeve may surround the transcatheter valve for sealing gaps between the valve and annuloplasty ring.

### CROSS REFERENCE TO RELATED APPLICATIONS

This application is a continuation of International Patent Application No. PCT/US2021/071152, filed Aug. 10, 2021, which claims the benefit of U.S. Patent Application No. 63/064,287, filed Aug. 11, 2020, the entire disclosures all of which are incorporated by reference for all purposes.

### TECHNICAL FIELD

The present disclosure relates generally to annuloplasty rings and, in particular, to expandable mitral annuloplasty rings.

### BACKGROUND

The heart is a hollow muscular organ having four pumping chambers separated by four heart valves: aortic, mitral (or bicuspid), tricuspid, and pulmonary. The mitral and tricuspid valves are defined by dense fibrous rings of collagen, each called an annulus, which forms a part of the fibrous skeleton of the heart. The annulus provides peripheral attachments for the two cusps or leaflets of the mitral valve (called the anterior and posterior cusps) and the three cusps or leaflets of the tricuspid valve. The native valve leaflets flex outward when the valve opens and their free edges come together or coapt in closure.

The free edges of the mitral leaflets connect to chordae tendineae from more than one papillary muscle. Mitral valve malfunction can result from the chordae tendineae (the chords) becoming stretched, and in some cases tearing. Also, a normally structured valve may not function properly because of an enlargement of or shape change in the valve annulus. This condition is referred to as a dilation of the annulus and generally results from heart muscle failure. In addition, the valve may be defective at birth or because of an acquired disease. From a number of etiologies, mitral valve dysfunction can occur when the leaflets do not coapt at peak contraction pressures. As a result, an undesired back flow of blood from the left ventricle into the left atrium can occur.

Various surgical techniques may be used to repair a diseased or damaged valve. A commonly used repair technique effective in treating incompetence is annuloplasty, which often involves reshaping or remodeling the annulus by attaching a prosthetic annuloplasty repair segment or ring thereto. For instance, the goal of a posterior mitral annulus repair is to bring the posterior mitral leaflet forward toward to the anterior leaflet to better allow coaptation. The annuloplasty ring is designed to support the functional changes that occur during the cardiac cycle: maintaining coaptation and valve integrity to prevent reverse flow while permitting good hemodynamics during forward flow.

Annuloplasty rings may be stiff, flexible or semi-rigid, and a “remodeling” annuloplasty ring typically has an inner core that resists conforming to the native annulus shape and instead forces the annulus to conform to it. Remodeling annuloplasty bands or rings are “generally rigid” or “semi-rigid” in that they will resist distortion when subjected to the physiological forces imparted thereon by the mitral valve annulus of an operating human heart. In this sense, “distortion” means substantial permanent deformation from a predetermined or manufactured shape (e.g., the band or ring will tend to return to its preset shape in use). A typical remodeling annuloplasty ring comprises an inner substrate or core of a metal such as a rod or multiple bands of nitinol, cobalt-chromium, stainless steel or titanium covered with a biocompatible fabric or cloth and perhaps silicone to allow the ring to be sutured to the fibrous annulus tissue.

Annuloplasty rings may have a variety of shapes in plan view, including closed or continuous oval, circular, D-shaped, or kidney-shaped, and open or discontinuous C-shaped, sometimes referred to as a band. Examples are seen in U.S. Pat. Nos. 5,041,130, 5,104,407, 5,201,880, 5,258,021, 5,607,471 and, 6,187,040. Most rigid and semi-rigid annular rings for the mitral valve have a kidney-like or D shape, with a curved posterior segment co-extensive with the posterior valve leaflet, and a somewhat straighter anterior segment co-extensive with the anterior valve leaflet. One successful semi-rigid ring is the Carpentier-McCarthy-Adams IMR ETlogix® annuloplasty ring sold by Edwards Lifesciences of Irvine, Calif. The IMR ETlogix ring is semi-rigid for remodeling purposes, and is intended to support the P2/P3 segments of the mitral valve that have dilated because of ischemia.

Nearly 20,000 patients undergo mitral valve repair each year and approximately 60,000 go untreated in the US. Currently, approximately 30% of mitral repairs for ischemic functional mitral regurgitation (FMR) result in reoperations.

Sometimes the need for complete mitral valve replacement may arise after a patient has already had an earlier annuloplasty ring repair. Rather than explanting the annuloplasty ring, a new prosthetic heart valve may be expanded directly within the ring (a so-called valve-in-ring procedure). For instance, U.S. Pat. No. 8,287,591 discloses various solutions for valve-in-ring systems, the entire disclosure of which is expressly incorporated herein by reference. Clinical trials have been undertaken as well, such as described in “Transfemoral Tricuspid Valve-in-Ring Implantation Using the Edwards Sapien XT Valve,” Circulation: Cardiovascular Interventions. 24315:8 (5 Mar. 2015). Valve-in-valve procedures are somewhat more common, such as described in U.S. Pat. No. 9,375,310, the entire disclosure of which is expressly incorporated herein by reference.

Despite some research into valve-in-ring and valve-in-valve procedures, there is a need for an annuloplasty ring that better accommodates a subsequently implanted expandable valve.

### SUMMARY

The present disclosure provides a mitral repair annuloplasty ring that will accommodate implantation of a transcatheter valve when there is a need for a reoperation of a patient that may not undergo open heart surgery. The mitral repair ring is a rigid/semi-rigid ring with features that allow the ring to conform to the geometry of a transcatheter valve when implanted. The mitral repair ring is based on existing mitral repair rings and will maintain the shape and relative rigidity needed for ischemic mitral valve repair. At the same time, the design of the new ring will allow for implantation of a transcatheter valve in the mitral position.

In an exemplary example, an annuloplasty ring for mitral valve remodeling and adapted for a valve-in-ring procedure comprises a solid inner core defining a continuous peripheral shape and a suture-permeable tubular interface surrounding the inner core. The inner core is formed of a semi-rigid remodeling material that will resist distortion when subjected to the physiological forces imparted thereon by the mitral valve annulus of an operating human heart. The peripheral shape is generally a D-shape with a substantially straight anterior side diametrically across from a more rounded posterior side with arcuate lateral sides therebetween, and the peripheral shape is oriented around a central axis at an intersection of a minor axis across the midpoints of the anterior and posterior sides and a major axis across the lateral sides. The inner core has radially thick and thin regions along discrete segments around the peripheral shape configured to enable different rates of bending when an internal dilatory force is introduced within the annuloplasty ring and the thick and thin regions are positioned to urge the D-shaped periphery to a more circular shape.

The inner core may have a first radially thick region at least along the posterior side relative to adjacent lateral sides. The first radially thick region is about twice the radial thickness of the lateral sides. The inner core may further have a second radially thick region along the anterior side relative to adjacent lateral sides, wherein the first and second radially thick regions are about twice the radial thickness of the lateral sides. In one example, the second radially thick region has a plurality of spaced radially-outwardly opening notches formed therein.

Another example of an annuloplasty ring for mitral valve remodeling adapted for a valve-in-ring procedure comprises a solid inner core defining a continuous peripheral shape and a suture-permeable tubular interface surrounding the inner core. The inner core is formed of a semi-rigid remodeling material that will resist distortion when subjected to the physiological forces imparted thereon by the mitral valve annulus of an operating human heart. The peripheral shape is generally a D-shape with a substantially straight anterior side diametrically across from a more rounded posterior side with arcuate lateral sides therebetween, and the peripheral shape is oriented around a central axis at an intersection of a minor axis across the midpoints of the anterior and posterior sides and a major axis across the lateral sides. The inner core further has radial slits opening to both inner and outer edges around the peripheral shape configured to enable different rates of bending along discrete segments of the inner core when an internal dilatory force is introduced within the annuloplasty ring, and the radial slits are positioned to urge the D-shaped periphery to a more circular shape.

There are preferably a first plurality of the radial slits opening outwardly and spaced along the anterior side, and a second plurality of the radial slits opening outwardly and spaced along the posterior side. There also may be plurality of the radial slits opening inwardly and spaced along the lateral sides. Each of the radial slits may be formed by a substantially straight channel extending radially from the respective edge of the inner core to an enlarged closed relief area, and each of the closed relief areas may be circular.

The inner core of the rings disclosed herein are preferably formed with a solid cross-section and made of a semi-rigid metal, such as nitinol, cobalt-chromium, stainless steel or a titanium alloy. The peripheral shape may be asymmetric across the minor axis, and the peripheral shape may be three-dimensional and bow down on one side of the minor axis from the anterior side.

The annuloplasty ring may further including at least one expansion joint around the peripheral shape along the inner core which enables a circumference of the inner core to expand when subjected to the internal dilatory force. A singular expansion joint may be located on an anterior side of the D-shaped inner core, or two expansion joints may be located on opposite sides of the D-shaped inner core generally along a major axis thereof.

A combination of an annuloplasty ring for mitral valve remodeling adapted for a valve-in-ring procedure and an expandable prosthetic heart valve is also disclosed. The annuloplasty ring may be configured in any of the aforementioned ways. The combination features an expandable prosthetic heart valve having an expandable support frame made to plastically expand with a generally circular cross-section when subjected to an outward dilatory force. The support frame is covered by a fabric cover surrounded by an attached sealing sleeve formed of a compressible or inelastic material. The heart valve has a contracted shape for advancement within the annuloplasty ring and an expanded shape sized to contact and expand the annuloplasty ring and convert the peripheral shape to a more circular shape, wherein the sealing sleeve expands into gaps between the heart valve and annuloplasty ring and is adapted to form radially thicker and thinner regions around a circumference of the heart valve.

In the aforementioned combination, the inner core may have radially thick and thin regions along discrete segments around the peripheral shape configured to enable different rates of bending when the internal dilatory force is introduced within the annuloplasty ring. Alternatively, the inner core has radial slits opening to both inner and outer edges around the peripheral shape configured to enable different rates of bending along discrete segments of the inner core when the internal dilatory force is introduced within the annuloplasty ring. The annuloplasty ring may further include at least one expansion joint around the peripheral shape of the annuloplasty ring which enables a circumference of the inner core to expand when subjected to the internal dilatory force.

The sealing sleeve desirably attaches to the fabric cover along one circumferential line so as to decouple axial foreshortening of the expandable support frame from the sealing sleeve. The sealing sleeve may comprise an annular bladder having inner and outer walls holding a fluid therein, and the annular bladder may be made of polyethylene terephthalate and the fluid is silicone. Or the sealing sleeve may comprise a solid material which is plastically deformable such as a hydrogel.

A further understanding of the nature and advantages of the disclosure will become apparent by reference to the remaining portions of the specification and drawings.

### BRIEF DESCRIPTION OF THE DRAWINGS

Features and advantages of the present disclosure will become appreciated as the same become better understood with reference to the specification, claims, and appended drawings wherein:

FIG. 1 is a perspective view of an exemplary mitral annuloplasty ring having a variable cross-sectional shape that expands subject to an internal dilatory force to a substantially circular peripheral shape;

FIGS. 2A-2C are plan and elevational views of the mitral annuloplasty ring of FIG. 1, and FIGS. 2D-2F are radial sectional views taken around the periphery thereof;

FIG. 3A is a schematic plan view of the mitral annuloplasty ring of FIG. 1 undergoing expansion from an internal dilatory force, and FIG. 3B is a plan view of the expanded mitral annuloplasty ring showing imposition of an expanded prosthetic heart valve therein;

FIG. 4A is a perspective view and FIG. 4B a plan view of an exemplary mitral annuloplasty ring having a variable cross-sectional shape that expands subject to an internal dilatory force to a substantially circular peripheral shape;

FIG. 5A is a perspective view and FIG. 5B a plan view of an exemplary mitral annuloplasty ring having a variable cross-sectional shape that expands subject to an internal dilatory force to a substantially circular peripheral shape;

FIG. 6 is a perspective view of another exemplary mitral annuloplasty ring core having a plurality of expansion slits therein that expands subject to an internal dilatory force to a substantially circular peripheral shape;

FIGS. 7A-7C are plan and elevational views of the mitral annuloplasty ring core of FIG. 6;

FIG. 8A is a schematic plan view of the mitral annuloplasty ring core of FIG. 6 undergoing expansion from an internal dilatory force, and FIG. 8B is a plan view of the expanded mitral annuloplasty ring core showing imposition of an expanded prosthetic heart valve therein;

FIG. 9A is an elevational view of a prosthetic heart valve having an external sealing sleeve crimped onto an expansion balloon, and FIG. 9B is a radial sectional view through the assembly showing the sealing sleeve;

FIG. 10 is an elevational view of the prosthetic heart valve with external sealing sleeve after expansion by the balloon;

FIG. 11 is a plan view of the expanded balloon and prosthetic heart valve of FIG. 10 imposed within the expanded mitral annuloplasty ring core of FIG. 3A and showing migration of the sealing sleeve into the gaps between the two;

FIG. 12 is a plan view of the expanded balloon and prosthetic heart valve of FIG. 10 imposed within the expanded mitral annuloplasty ring core of FIG. 8A and showing migration of the sealing sleeve into the gaps between the two;

FIG. 13 is a perspective view of an alternative mitral annuloplasty ring core having an expansion joint located on an anterior side thereof, and FIG. 14 is a reverse perspective view showing an expansion member exploded from within an expansion slot;

FIG. 15A is a schematic plan view of the mitral annuloplasty ring core of FIG. 13 undergoing expansion from an internal dilatory force, and FIG. 15B is a plan view of the expanded mitral annuloplasty ring core showing imposition of an expanded prosthetic heart valve therein;

FIG. 16A is a plan view of a still further mitral annuloplasty ring core having two expansion joints opposite each other on lateral sides, and FIG. 16B is a plan view of the expanded mitral annuloplasty ring core showing imposition of an expanded prosthetic heart valve therein;

FIG. 17 is a plan view of the expanded balloon and prosthetic heart valve of FIG. 10 imposed within the expanded mitral annuloplasty ring core of FIG. 13 and showing migration of the sealing sleeve into the gaps between the two; and

FIG. 18 is a plan view of the expanded balloon and prosthetic heart valve of FIG. 10 imposed within the expanded mitral annuloplasty ring core of FIG. 16A and showing migration of the sealing sleeve into the gaps between the two.

### DETAILED DESCRIPTION

The present application discloses a mitral annuloplasty ring that . The term “ring” is used here since the implant is a closed or continuous ring, although in some contexts such implants are also termed “bands.” A preferred plan view shape of the disclosed rings is kidney or D-shaped so as to conform to the peripheral shape of the usual mitral annulus.

The right ventricle RV and left ventricle LV are separated from the right atrium RA and left atrium LA, respectively, by the tricuspid valve TV and mitral valve MV; e.g., the atrioventricular valves. Though correction of the mitral annulus is the primary focus of the present application, it should be understood that certain characteristics of the annuloplasty bands described herein may equally be used to treat the tricuspid valve TV, and thus the claims should not be constrained to the mitral band unless expressly limited.

The term “axis” in reference to the illustrated annuloplasty bands, and other non-circular or non-planar bands, refers to a line generally through the centroid of the band or ring periphery when viewed in plan view. “Axial” or the direction of the “axis” can also be viewed as being parallel to the average direction of blood flow within the valve orifice and thus within the band when implanted therein. Stated another way, an implanted mitral band or ring orients about a central flow axis aligned along an average direction of blood flow through the mitral annulus from the left atrium to the left ventricle.

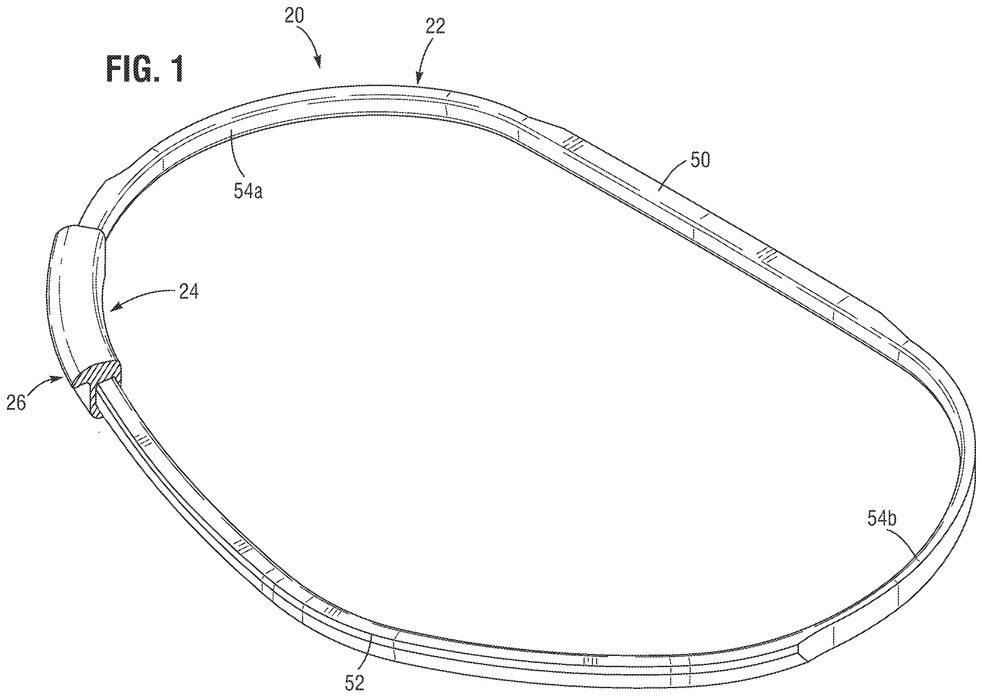

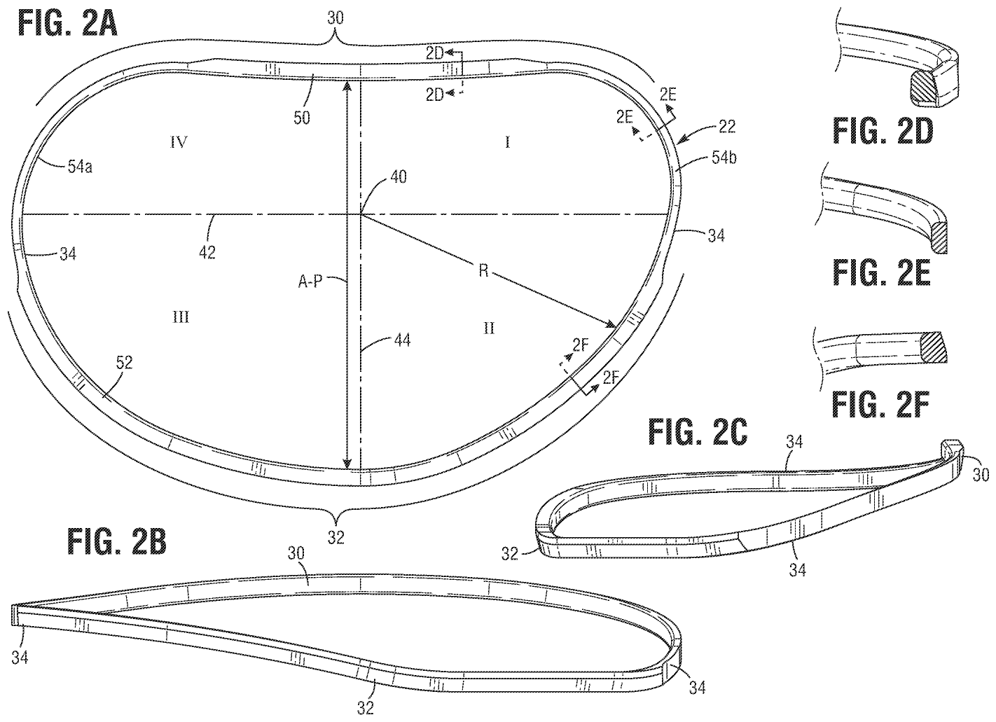

FIG. 1 is a perspective view of an exemplary mitral annuloplasty ring 20 having a variable cross-sectional shape, and FIGS. 2A-2C are plan and elevational views thereof. The annuloplasty ring 20 is configured to expand subject to an internal dilatory force to a substantially circular peripheral shape.

In a preferred example, the ring construction includes a relatively rigid inner core 22 formed in a continuous ring and surrounded by a suture-permeable tubular interface 24, only a segment of which is shown in cutaway. In the illustrated example, the inner core 22 is a solid cross-section, with a greater axial dimension than radial dimension. The core material is a “semi-rigid” metal meaning it will flex but is stiff enough to resist permanent distortion when subjected to the physiological forces imparted thereon by the mitral valve annulus of an operating human heart. In other words, the annulus conforms to the ring instead of the other way around. Preferably, the material of the inner core 22 is an elastic-plastic metal such as nitinol, cobalt-chromium, stainless steel or titanium alloy. This applies to all of the core examples disclosed herein, whether or not there are expansion joints or not.

The tubular interface 24 may include an elastomeric sleeve closely surrounding the entire core and a fabric outer cover (not shown), for example, a polyethylene terephthalate (PET) fabric cover. In the preferred example, the elastomeric sleeve, which may be silicone rubber, is molded to have a radially outwardly-extending flange 26 to facilitate suturing of the ring 20 to the mitral annulus. The ring 20 may be secured with sutures, staples, or other such devices to an inside ledge of the mitral annulus. In a typical procedure, an array of sutures is anchored through the annulus and then threaded through corresponding locations around the interface on the outside of the ring 20, and then the ring is parachuted down the suture array to be seated at the annulus before tying off the sutures.

With reference now to FIGS. 2A-2C, the D-shaped ring core 22 defines a substantially straight anterior side 30 opposite a more curved posterior side 32, with arcuate lateral sides 34 therebetween. The ring core 22 forms the primary structural component of the annuloplasty ring 20, and thus defines the rounded D-shape of the ring as mentioned. As with many mitral annuloplasty rings, the inner core 22 is arranged around a central flow axis 4o at the intersection of a major axis 42 and a minor axis 44. The dimension across the inner core 22 along the minor axis 44 is denoted the A-P dimension, for anterior-posterior.

The annuloplasty ring 20 may be three-dimensional with an upward bow in the anterior side 30 and a downward bow on the posterior side 32, as seen in FIGS. 2B and 2C. The ring 20 as seen from above in FIG. 2A may be segregated into four quadrants (I-II-III-IV) as defined by the major and minor axes 42, 44 starting from the upper right and going clockwise. The inner core 22 begins to dip downward at a point in the first quadrant and climbs back up in the third quadrant. (It should be noted that a majority of the ring 20 is below the upper middle of the anterior side 30, and the asymmetric downward bow in quadrants I and II is relative to the mirror-image quadrants III and IV on the opposite side.) Additionally, the “radial” distance R between the inner core 22 and the central flow axis 40 is reduced in the second quadrant and a portion of the first quadrant relative to the opposite side across the minor axis 44. This asymmetry across the minor axis 44 in plan and elevational views is believed to better correct for certain pathologies such as ischemic mitral regurgitation (IMR). The shape of the ring 20 is similar to that of the Carpentier-McCarthy-Adams IMR ETlogix® annuloplasty ring available from Edwards Lifesciences of Irvine, Calif.

In contrast to earlier mitral annuloplasty rings, the ring core 22 includes thin and thick regions that help determine a final expanded shape upon application of an internal dilatory force. More particularly, the thin and thick regions along discrete segments around the peripheral shape of the ring core 22 cause different rates of bending when an internal dilatory force is introduced within the annuloplasty ring 20 to urge the D-shaped periphery to a more circular shape. The term “internal dilatory force” means an outward expansion force from within the ring 20. Such an expansion force may be applied by a balloon by itself, or a by a balloon expanding a prosthetic heart valve outward into contact with the mitral annulus and pre-implanted annuloplasty ring in a valve-in-ring procedure. It should be understood that the magnitude of an internal dilatory force is significantly greater than physiological forces imparted to the ring by the natural cyclic systolic-diastolic motion of the heart. For instance, the magnitude of an internal dilatory force required to expand a ring core as disclosed herein is greater than 1 atm (760 mm Hg).

As explained above, the inner core 22 comprises a continuous elastic-plastic metal such as nitinol, cobalt-chromium stainless steel or titanium alloy. Typically, such structures present a relatively rigid peripheral shape which is difficult to expand. Moreover, the plan view shape of the ring core 22, as seen in FIG. 2A is a rounded D-shape so as to mimic the shape of the native mitral annulus. However, expandable prosthetic heart valves used in valve-in-ring procedures have substantially cylindrical exterior profiles which do not match the rounded D-shape. As a result, gaps are formed around the expanded heart valve which may lead to regurgitation.

With reference back to FIG. 1, and also with reference to FIGS. 2A-2F, the inner core 22 includes a first radially thick region 50 located on the anterior side 30, a second radially thick region 52 located on the posterior side 32, and radially thin regions 54a, 54b located at the arcuate lateral sides 34. The sectional shapes and relative sizes of these regions 50, 52, 54 are shown in FIGS. 2D-2F. It should be noted that although only one of the thin regions 54b is shown in section, the other thin region 54a may be identical. The inner core 22 has a solid generally rectangular cross-section, in that there are generally four surfaces; namely, top, bottom, inner and outer faces. In the thick regions 50, 52 shown in FIGS. 2D and 2F, at the anterior and posterior sides 30, 32, respectively, the outer face may be angled inward toward the upper corner with the top and bottom faces being generally parallel and at roughly 90° angles to the inner face. The thin region 54a cross-section of FIG. 2E at the lateral sides 34 is thinner radially than the thick regions 50, 52, but the outer face is roughly parallel to the inner face. In all cases the corners between the inner face and the top and bottom faces are rounded to help avoid any abrasion of the valve leaflets moving within the ring. In one example, the axial dimension of the ring core 22 is substantially constant around the periphery, though the radial thickness at the lateral sides 34 is about one half of the radial thickness at the anterior and posterior sides 30, 32. In one example, the boundary between the thick and thin regions is tapered rather than being abrupt.

In absolute terms, a typical ring radial thickness is about 0.05 inches (1.27 mm). The thin regions may be reduced by half, to about 0.025 inches (0.64 mm). Although a 50% reduction from the thick to the thin regions is considered effective, the thickness reduction may be more or less than 50%, primarily depending on how much deformation is desired. Therefore, a ratio of the radial dimension between the thin and thick regions is preferably about 50%, but may be down to 25%, and up to 80%. Furthermore, the ring cores shown have discrete spans of either thick or thin regions in a binary pattern, whereas more spans of intermediate thicknesses may be utilized, such as by providing a constantly varying periphery with no abrupt changes whatsoever. One of skill in the art will understand that the ring cores may be customized to perform in innumerable ways when stressed by altering the thickness around the periphery.

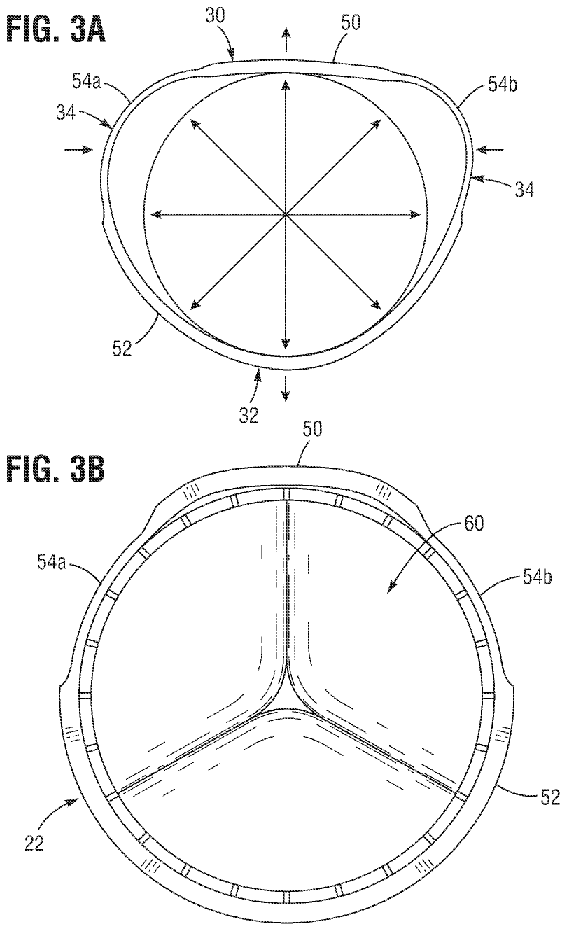

Placement of the thick and thin regions 50, 52, 54 around the periphery of the inner core 22 is designed to encourage the core to expand into a circular shape. For instance, FIG. 3A is a schematic plan view of the mitral annuloplasty ring 20 undergoing expansion from an internal generally axi-symmetric dilatory force, represented by a circle with expansion arrows to simulate a balloon or balloon and expanding prosthetic heart valve. Because the dimension along the minor axis 44 of the ring 20 is less than the dimension across the major axis 42, the expanding structure initially contacts the anterior and posterior sides 30, 32, thus forcing them apart. Because these sides 30, 32 incorporate the thick regions 50, 52, they do not deform as much as the thin regions 54a, 54b. Moreover, due to the relatively greater flexibility of the thin regions 54a, 54b, the lateral sides 34 tend to straighten out and move together, as seen. Eventually, the entire structure of the core 22 becomes more circular. In other words, the different thickness regions 50, 52, 54 around the periphery of the ring core 22 experience different rates of bending to urge the D-shaped periphery to a more circular shape.

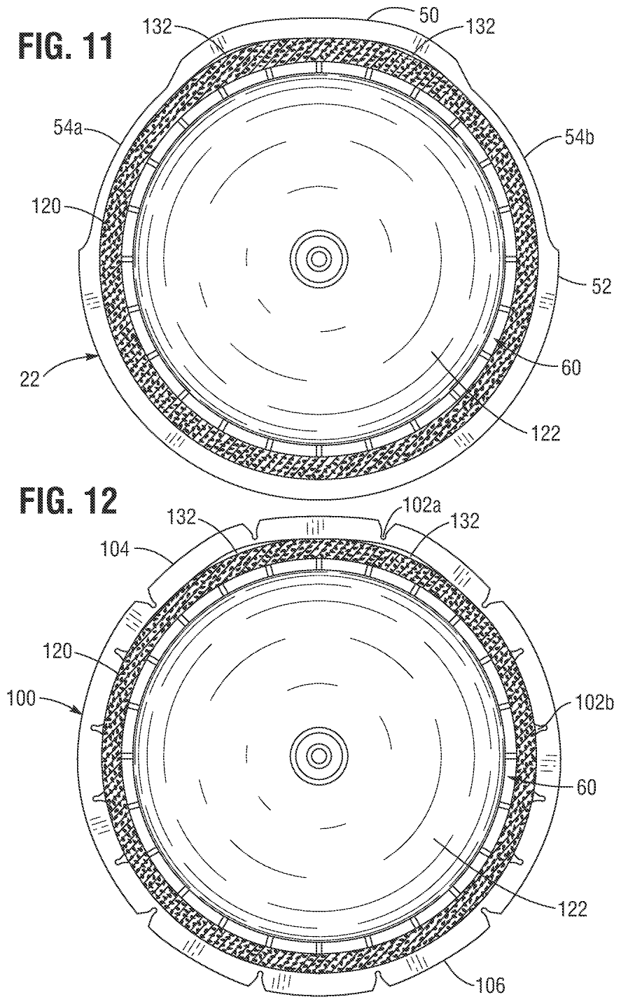

FIG. 3B is a plan view of the expanded mitral annuloplasty ring 20 (with the outer suture-permeable tubular interface 24 removed for clarity) showing imposition of an expanded prosthetic heart valve 60 therein. The heart valve 60 may be self-expanding or balloon-expanding. If the former, a dilatory balloon may be first used to expand the annulus and annuloplasty ring 20, and then the heart valve 60 is advanced and permitted to expand within the ring. If the latter, the dilatory balloon expands the heart valve 60 directly outward into contact with the annulus and the annuloplasty ring 20. Although the illustration is a simulation, and the ultimate peripheral shape of the annuloplasty ring 20 may not be entirely circular, those of skill in the art will understand that the presence of the more flexible thin regions 54a, 54b enables the core 22 to more faithfully expand toward a circular shape. This minimizes gaps between the annuloplasty ring 20 and the heart valve 60 therewithin, thus minimizing any potential regurgitation therebetween. Moreover, the exterior of the heart valve 60 may be provided with sealing structures such as fabric skirts and the like to further reduce any regurgitation, as described below with reference to FIG. 11.

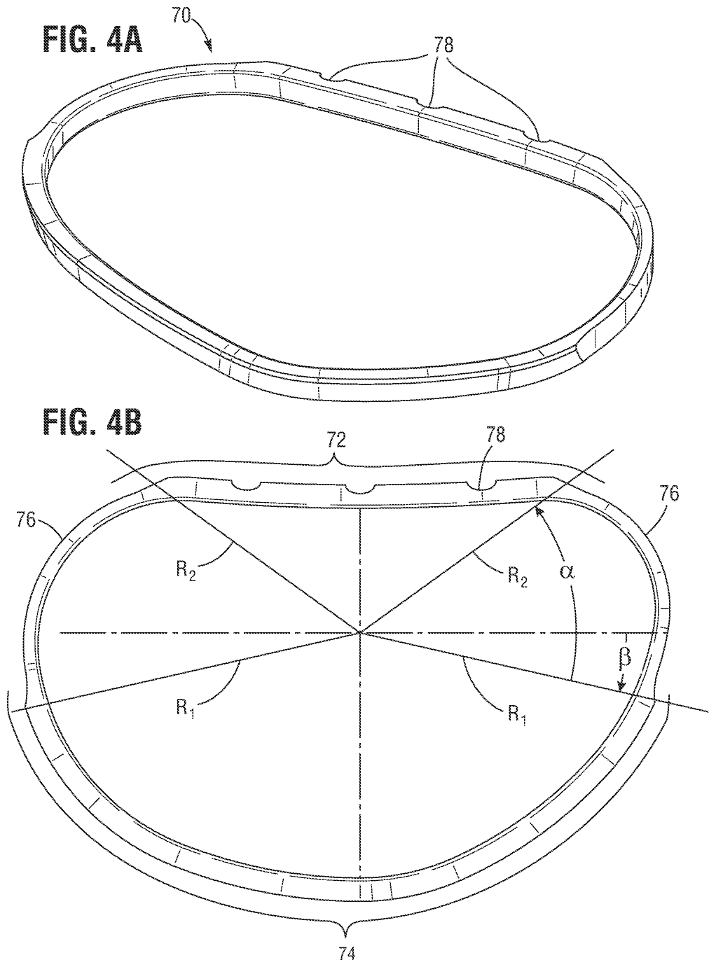

FIG. 4A is a perspective view and FIG. 4B a plan view of an exemplary mitral annuloplasty ring core 70 similar to the ring core 22 shown in FIGS. 1-2 with a variable cross-sectional shape that expands subject to an internal dilatory force to a substantially circular peripheral shape. The annuloplasty ring comprises the inner core 70 as shown and typically has a suture-permeable tubular interface surrounding the core for anchoring the ring at the annulus which is not shown for clarity.

As with the earlier core 22, the ring core 70 has an asymmetric D-shape in plan view with a straighter anterior side 72 opposite a more rounded posterior side 74, and lateral sides 76 therebetween. A radial thickness of the ring core 70 is greater along both the anterior side 72 and the posterior side 74 than at the lateral sides 76. In particular, a first radially thick region extends along the posterior side 74 relative to adjacent lateral sides 76, and a second radially thick region extends along the anterior side 72 also relative to adjacent lateral sides. The relative thicknesses may be as described above with respect to the core 22. To help the thickened anterior side 72 more easily conform to an expanding cylindrical dilatory force, a series of outwardly opening notches 78 are provided. In particular, three generally semi-circular notches 78 are evenly spaced along the anterior side 72. These notches reduce the bending strength of the anterior side 72, thus facilitating its transformation from the substantially straight configuration shown to a more rounded arcuate shape after expansion.

FIG. 4B shows an exemplary span for the first and second radially thick regions. As described above, the D-shaped mitral ring core 70 is arranged about perpendicular major and minor axes that intersect at a central axis. Although not circular, the ring core 70 is continuous and thus extends 360° around the central axis, and various angular spans may be described using such nomenclature. The first radially thick region extending along the posterior side 74 spans between the border radial lines R₁, and the second radially thick region extending along the anterior side 72 spans between the border radial lines R₂. The radial lines R₁ and R₂ on each lateral side are separated by a first angle α, and a second angle β is formed between the radial lines R₁ and the major axis. The first radially thick region extending along the posterior side 74 spans a greater angle than does the second radially thick region extending along the anterior side 72, and is preferably between 1.5-2 times as long.

The total span of the first and the second radially thick regions equals 360°−2(α). For the purpose of an illustrative example, the angle α=45°, and so the total span of the first and the second radially thick regions equals 270° (360°−90°). In one example, the angle β=10°, and so the span of the first radially thick region equals 160° (180°−20°). Consequently, the span of the second radially thick region equals 90° (270°−160°), and the span of the second radially thick region is about 1.8 times the span of the second radially thick region. Many variations on these spans are contemplated, and the range of angles are desirably 40°>α>60° and 5°>β>20°. It should be understood that the spans and exemplary angular ranges described with respect to FIG. 4B apply to all of the ring cores disclosed herein having radially thick and thin regions.

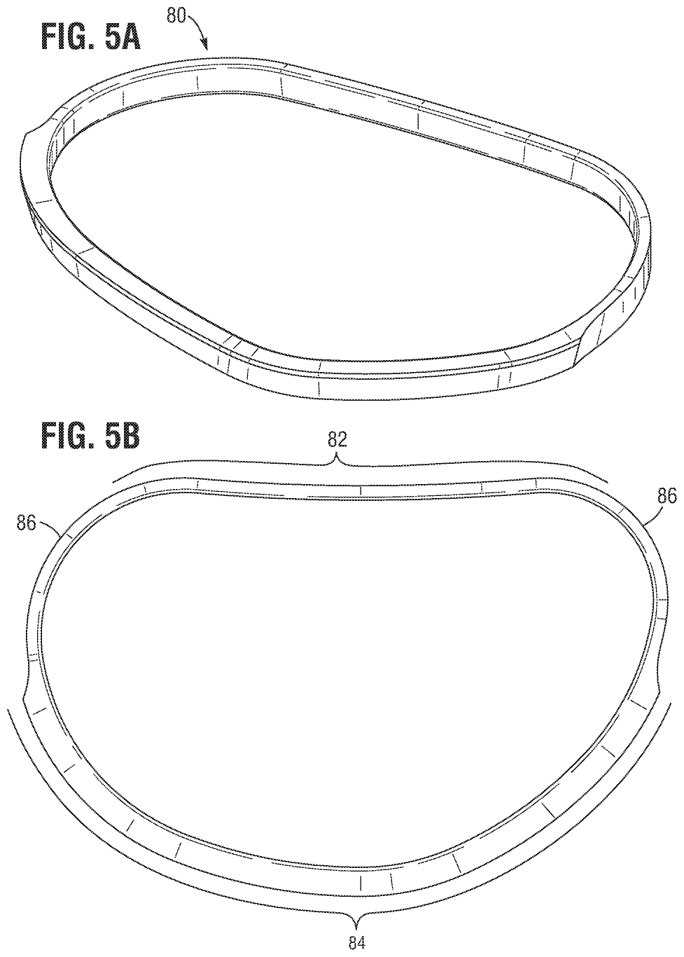

FIG. 5A is a perspective view and FIG. 5B a plan view of a further exemplary mitral annuloplasty ring core 80 having a variable cross-sectional shape that again is configured to expand subject to an internal dilatory force to a substantially circular peripheral shape. Like the ring core 70, the ring core 80 has an asymmetric D-shape in plan view with a straighter anterior side 82 opposite a more rounded posterior side 84, and lateral sides 86 therebetween. A radial thickness of the ring core 80 is greater along just the posterior side 84 than along the anterior side 82 and the lateral sides 86. This helps ensure that the anterior side 82 easily conforms to the cylindrical expansion force imparted by a balloon, or balloon acting on a prosthetic heart valve in a valve-in-ring procedure. Once again, the relative thicknesses of the sections around the ring core 80 may be the same as described above with respect to the ring core 22.

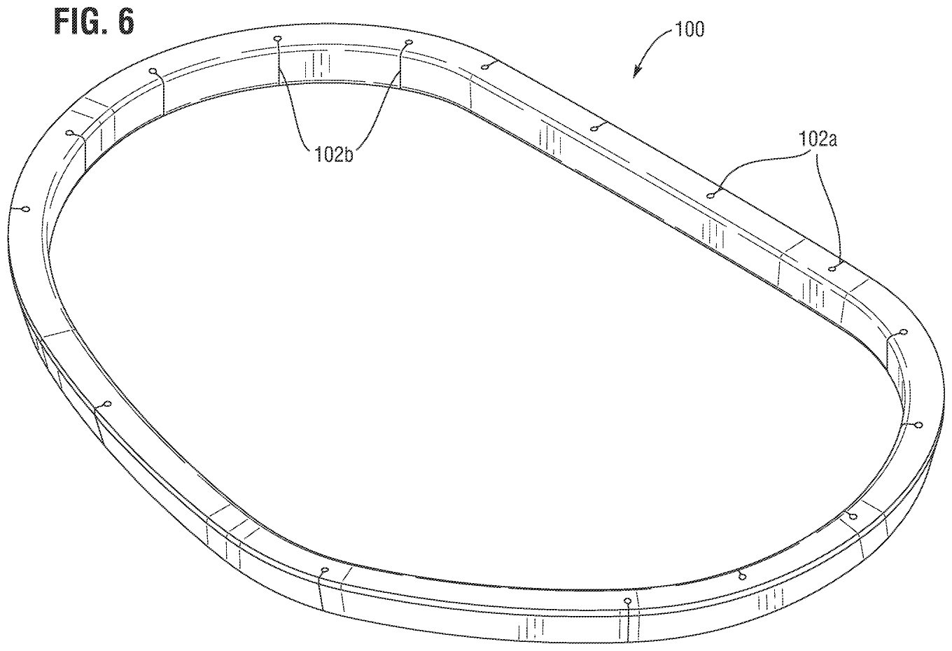

FIG. 6 is a perspective view of a different exemplary mitral annuloplasty ring core 100 having a plurality of expansion slits 102a, 102b therein. The slits 102a, 102b enable the ring core 100 to expand subject to an internal dilatory force to a substantially circular peripheral shape, as will be seen. Although not shown, a suture-permeable tubular interface typically surrounds the ring core 100 for passage of sutures anchoring the ring to the annulus.

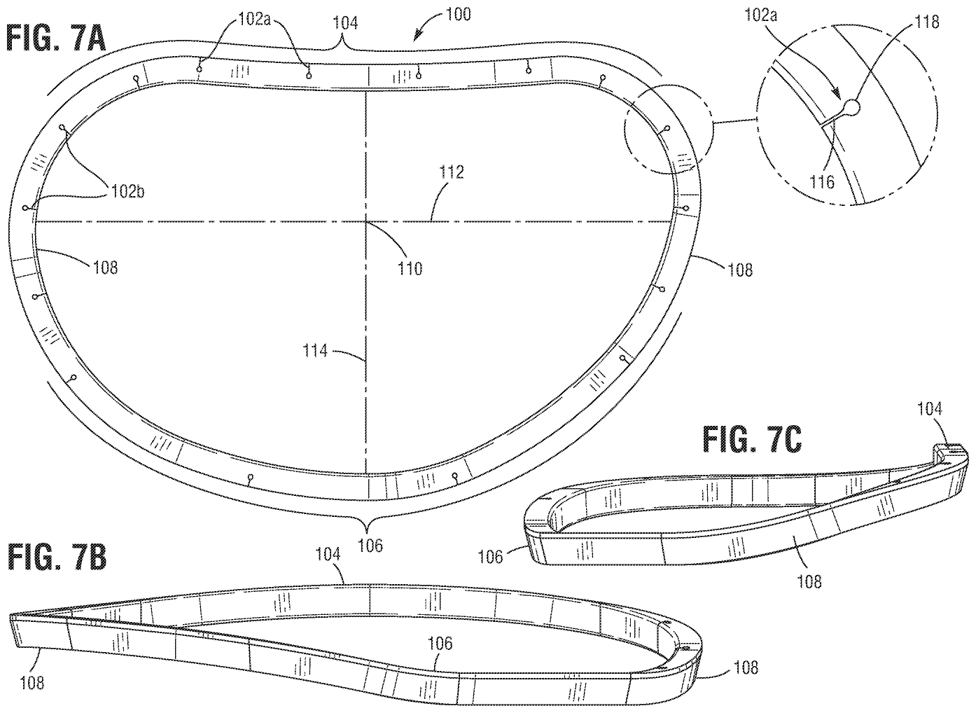

FIGS. 5A-5C are plan and elevational views of the mitral annuloplasty ring core 100 illustrating the aforementioned asymmetric D-shaped periphery. As before, a generally straight anterior side 104 lies opposite a curved posterior side 106, with lateral sides 108 therebetween. Once again, the ring core 100 defines a central flow axis 110 at the intersection of a major axis 112 and a minor axis 114. The anterior side 104 is at the highest axial elevation, with the posterior side 106 being lower, and quadrant II (see FIG. 2A) being at the lowest elevation. In plan view as seen in FIG. 7A, the ring core 100 around a portion of quadrants I and II is pulled in somewhat relative to the opposite quadrants diametrically across the minor axis 114.

Now with reference to FIG. 7A, the ring core 100 is seen to have exterior slits iota spaced around the outside periphery and interior slits 102b spaced around the inside periphery. One of the interior slits 102b is seen in large to the right and comprises a substantially straight channel 116 extending radially from the inside periphery to an enlarged closed relief area 118. In the illustrated example, the closed relief area 118 is substantially circular, but may be formed in other shapes. In the illustrated example, there are a series of exterior slits 102a spaced along both the anterior side 104 and the posterior side 106, with interior slits 102b spaced around both lateral sides 108. More specifically, there are four exterior slits 102a spaced along both the anterior and posterior sides 104, 106, and four interior slits 102b spaced on both lateral sides 108. The number and spacing of the interior and exterior slits 102a, 102b may be modified from the pattern shown, but the placement of the interior and exterior slits relative to the periphery of the ring will generally remain the same so as to encourage a more circular expanded shape. Namely, the radial slits 102a, 102b opening to both inner and outer edges enable different rates of bending along certain segments of the ring core 100 that results in an expanded core being more circular than a core without the slits.

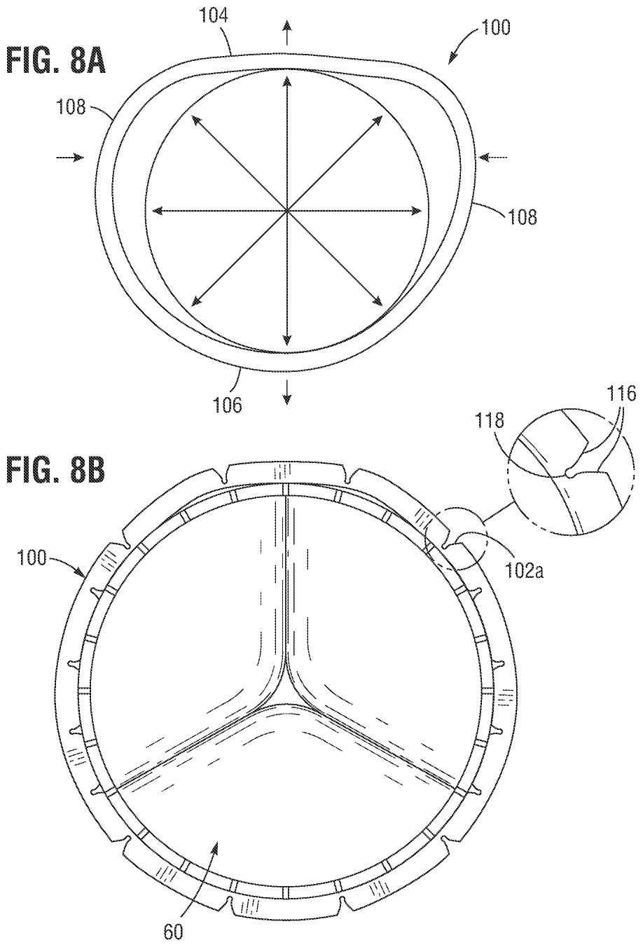

FIG. 8A is a schematic plan view of the mitral annuloplasty ring core 100 undergoing expansion from an internal dilatory force, and FIG. 8B is a plan view of the expanded mitral annuloplasty ring core showing imposition of an expanded prosthetic heart valve 60 therein. In general, as indicated by the deformation arrows in FIG. 8A, the anterior and posterior sides 104, 106 are urged apart, while the lateral sides 108 constrict inward toward each other.

FIG. 8B illustrates the effect of the internal dilatory force on one of the external slits 102a. In particular, the sides of the channel 116 spread apart and the enlarged relief area 118 provides a stop to further crack propagation. That is, the material of the ring core 100 is desirably nitinol, cobalt-chromium, stainless steel or titanium, and the rounded relief area 118 helps prevent complete severing of the ring core at that point in contrast to a slit without the relief area which might continue through the material.

Each of the external and internal slits 102a, 102b undergoes the spreading apart, though some may spread more than the others. The depth of the slits 102a, 102b also has an effect on how wide the slits spread at any one point. In one example, each of the slits 102a, 102b extends about half-way radially through the ring core cross-section. In general, because the anterior and posterior sides 104, 106 are forced radially outward, the exterior slits 102a allow the ring core 100 along the sides to constrict inward toward the ultimately circular heart valve 60. In contrast, as the lateral sides 108 contract inward they become somewhat straighter which is accommodated by expansion of the interior slits 102b. Another way to look at it is that the anterior and posterior sides 104, 106 conform more around the curve of the expanding circular dilatory force, while the posterior sides 108 pull inward to reduce the gaps between them and the expanding circular shape. Those of skill in the art will understand that careful placement, spacing and sizing of the exterior and interior slits 102a, 102b can be used to affect the final expanded shape of variously-shaped ring cores so as to be a circular as possible.

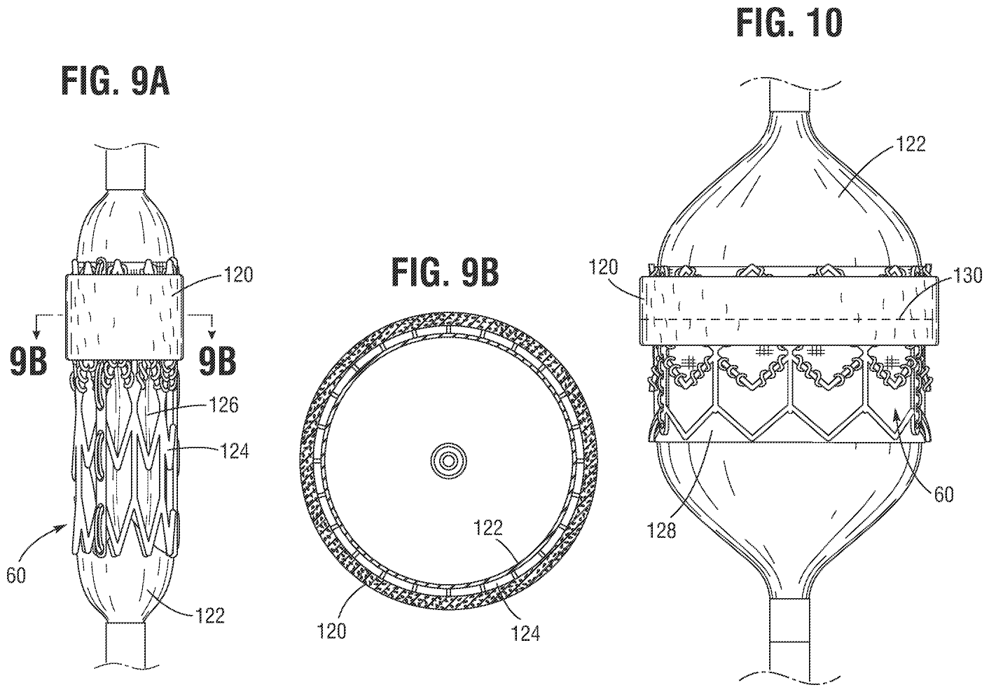

FIG. 9A is an elevational view of a prosthetic heart valve 60 having an external sealing sleeve 120 and crimped onto an expansion balloon 122, and FIG. 9B is a radial sectional view through the assembly showing the external sealing sleeve. The prosthetic heart valve 60 typically includes a self-or balloon-expandable frame 124 supporting a plurality of flexible bioprosthetic leaflets 126 in a flow orifice therein. In the illustrated example, the frame 124 is balloon-expandable and may be crimped around the expansion balloon 122 for subsequent delivery and expansion at an implantation site.

The external sealing sleeve 120 may comprise a compressible or flowable (inelastic) material, or may be configured as an annular bladder having inner and outer walls holding a fluid therein. Preferably, the external sealing sleeve 120 includes a flexible biocompatible bladder of, for instance, polyethylene terephthalate (PET), with a filling of deformable material such as silicone which may be cured to hold a shape. In another configuration, the sealing sleeve 120 is a solid material (no bladder) which is plastically deformable, having a putty-like quality, such as a hydrogel, that may compress unevenly around the heart valve 60 and end up thicker in some areas than others. Whatever way the sealing sleeve 120 is configured, it is desirably secured around the exterior of the prosthetic heart valve 60. Preferably, the sealing sleeve 120 has an axial dimension sufficient to extend around the part of the prosthetic heart valve 60 containing the inflow end of the bioprosthetic leaflets 126. For instance, the prosthetic heart valve 60 may have a crimped axial length of about 30 mm, while the sealing sleeve 120 has an axial length of about 10 mm. When the prosthetic heart valve 60 expands, both it and the sealing sleeve 124 shorten somewhat in the axial direction, but the proportional axial lengths remain approximately the same.

FIG. 10 is an elevational view of the prosthetic heart valve 60 with external sealing sleeve 120 after expansion by the balloon 122. As mentioned, the heart valve 60 and the sealing sleeve 120 undergo some axial foreshortening, though if the sealing sleeve 120 is not attached along its entire length it may not shorten proportionally as much. For instance, a prosthetic heart valve 60 may shorten from 30 mm to 20 mm, while the sealing sleeve 120 may only shorten from 10 mm to 8 mm. Preferably, the expandable frame 124 also has a fabric skirt or layer 128 extending inside, outside or both to further help prevent paravalvular leakage and to which the sealing sleeve 120 may be secured, such as via a single line of stitching 130, or at least stitching concentrated along one circumferential line to decouple the axial foreshortening of the expandable frame 124 from the sealing sleeve 120.

FIG. 11 is a plan view of the expanded balloon 122 and prosthetic heart valve 60 of FIG. 10 imposed within the expanded mitral annuloplasty ring core 22 of FIG. 3A. As mentioned above with reference to FIG. 3B, the presence of the more flexible thin regions 54a, 54b enables the core 22 to more faithfully expand toward a circular shape and minimize gaps 132 between the annuloplasty ring 20 and the heart valve 60. To further prevent paravalvular leaking, the sealing sleeve 120 compresses unevenly and migrates or flows into the gaps 132 that may remain between the two structures. That is, the radial thickness of the sealing sleeve 120 will vary around the circumference of the heart valve 60 after expansion due to the compressibility and/or plastic nature of the material. Depending on the size of the gaps 132 and configuration of the sealing sleeve 120 most of not all of the potential leaks will be plugged.

Likewise, FIG. 12 is a plan view of the expanded balloon 122 and prosthetic heart valve 60 of FIG. 10 imposed within the expanded mitral annuloplasty ring core 100 of FIG. 8A. As mentioned above with reference to FIG. 8B, the presence of the external slits 102a enable the core 100 to more faithfully expand toward a circular shape and minimize gaps 132 between the annuloplasty ring and the heart valve 60. To further prevent paravalvular leaking, the sealing sleeve 120 compresses unevenly and migrates into the gaps 132 that may remain between the two structures. Again, depending on the size of the gaps 132 and configuration of the sealing sleeve 120 most of not all of the potential leaks will be plugged.

Up to this point the annuloplasty rings configured to expand to substantially circular peripheral shapes have had solid or otherwise contiguous structural cores, albeit with thick/thin regions or strategically placed slits. The cores expand from D-shaped to more circular shapes, but the total circumferential length remains the same. An alternative core construction with expansion segments may also be utilized which expands to a more circular shape while also undergoing an increased circumferential length. The following examples are illustrative. Although not shown, these expandable core constructions may be combined with the thick/thin regions or have internal and/or external slits as described above.

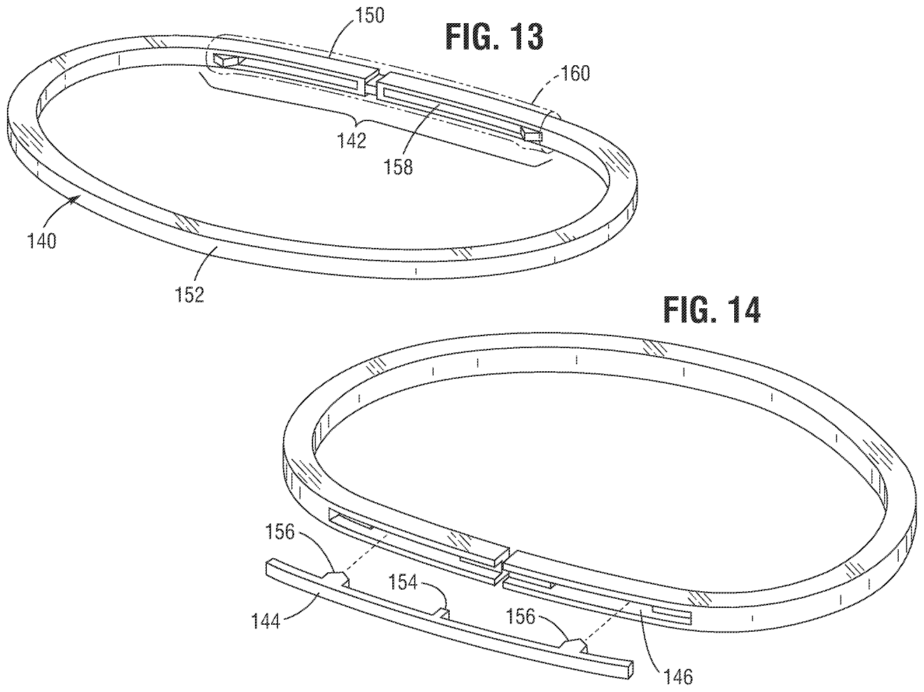

FIG. 13 is a perspective view of an alternative mitral annuloplasty ring core 140 having an expansion joint 142 located on an anterior side thereof, and FIG. 14 is a reverse perspective view showing an expansion insert 144 exploded from within an expansion slot 146.

The inner core 140 may be shaped substantially the same as described above with a rounded D-shaped periphery defining a straight anterior side 150 opposite a more curved posterior side 152. The anterior side 150 of the inner core 140 has the elongated outwardly-opening circumferential slot 146 which closely receives the expansion insert 144. The expansion insert 144 has a thin arcuate configuration with a central boss 154 extending radially inward as well as a pair of expansion-limiting bosses 156 also extending radially inward from opposite ends. As seen in FIG. 13, the anterior side 150 also includes on its inner periphery a pair of spaced apart short slots 158 through which the expansion-limiting bosses 156 project.

The anterior side 150 is bifurcated at its midpoint such that the two abutting ends may be separated due to an internal dilatory expansion force, as described above. In the relaxed or contracted configuration of the inner core 140, the abutting ends of the anterior side 150 prevent the ring circumference from reducing below that of its relaxed state. This allows the ring having the ring core 140 to function in a remodeling capacity, as with the continuous unbroken ring cores of FIGS. 1-6. As the free ends of the anterior side 150 separate, the short slots 158 slide outward over the expansion insert 144 until the expansion-limiting bosses 156 reach the other end of the short slots, which prevents further expansion. Each of the expansion-limiting bosses 156 has a triangular inner end which closely engages an inside of a compression sleeve 160 provided around the expansion joints. The compression sleeve 160 helps prevent premature expansion of the inner core 140 prior to application of a dilatory force such as from a balloon or expanding heart valve, and may be formed of a heat-shrinkable polyethylene terephthalate (PET).

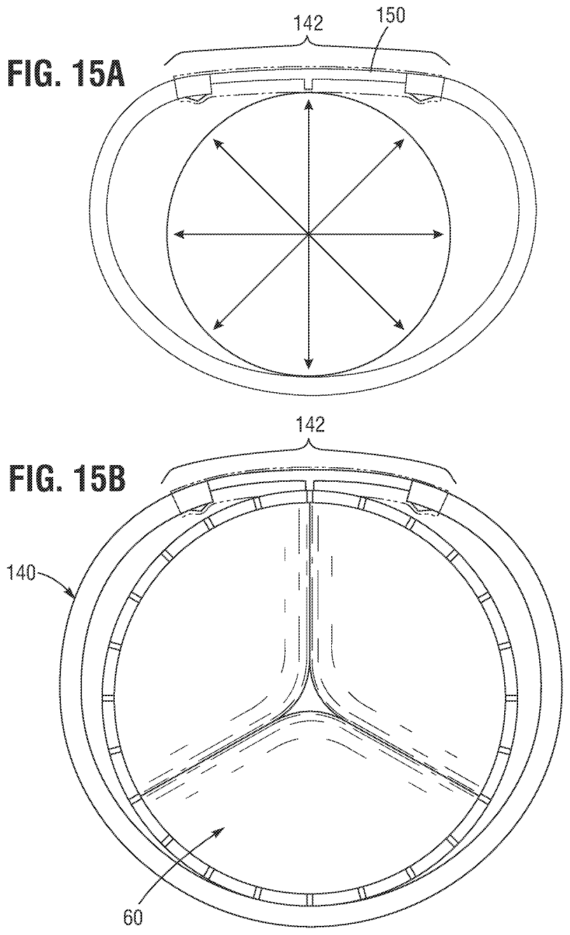

FIG. 15A is a schematic plan view of the mitral annuloplasty ring core 140 undergoing expansion from an internal dilatory force, while FIG. 15B is a plan view showing imposition of an expanded prosthetic heart valve 60 of generally circular cross-section therein. As with the earlier-described ring cores, the ring core 140 expands along the anterior-posterior dimension (e.g., along the minor axis), while the lateral sides constrict inward. Because the expansion joint 142 is centered in the straight anterior side 150, the overall peripheral shape of the ring core 140 becomes more circular for conforming better around the prosthetic heart valve 60, as seen in FIG. 15B. Once again, any gaps that still remain between the outwardly extending prosthetic heart valve 60 are minimized and may be filled with a fabric covering around the heart valve or a conformable cuff, as explained below.

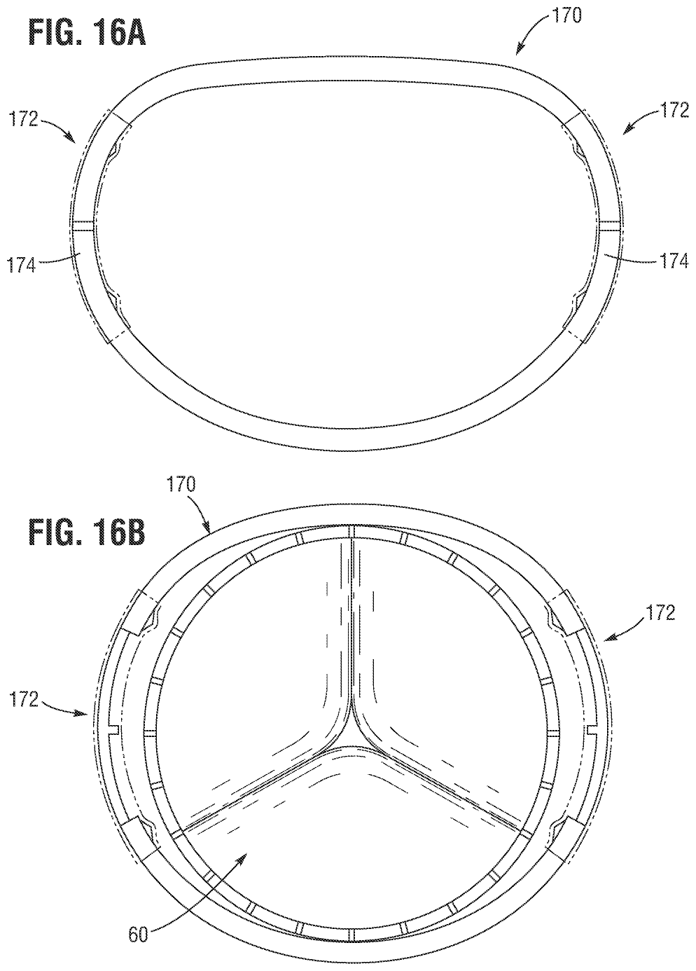

FIG. 16A is a plan view of a still further mitral annuloplasty ring core 170 having two expansion joints 172 opposite each other on lateral sides 174, and FIG. 16B is a plan view of the expanded mitral annuloplasty ring core showing imposition of an expanded prosthetic heart valve 60 therein. The shape of the ring core 170 will not be described again, but can be presumed to be D-shaped in plan view and three-dimensional as described above. The expansion joints 172 may be similar to the expansion joint 142 described above with respect to FIGS. 13-15.

As a balloon or balloon within the prosthetic heart valve 6o expands, the expansion joints 172 spread apart which tends to elongate and radially constrict the lateral sides 174. The resulting peripheral shape of the ring core 170 is more circular, as shown. This helps conform the cylindrical heart valve 60 within the ring core 170, thus reducing the leakage therebetween. Again, fabric skirts or cuffs may be provided on the heart valve 60 to further reduce leakage.

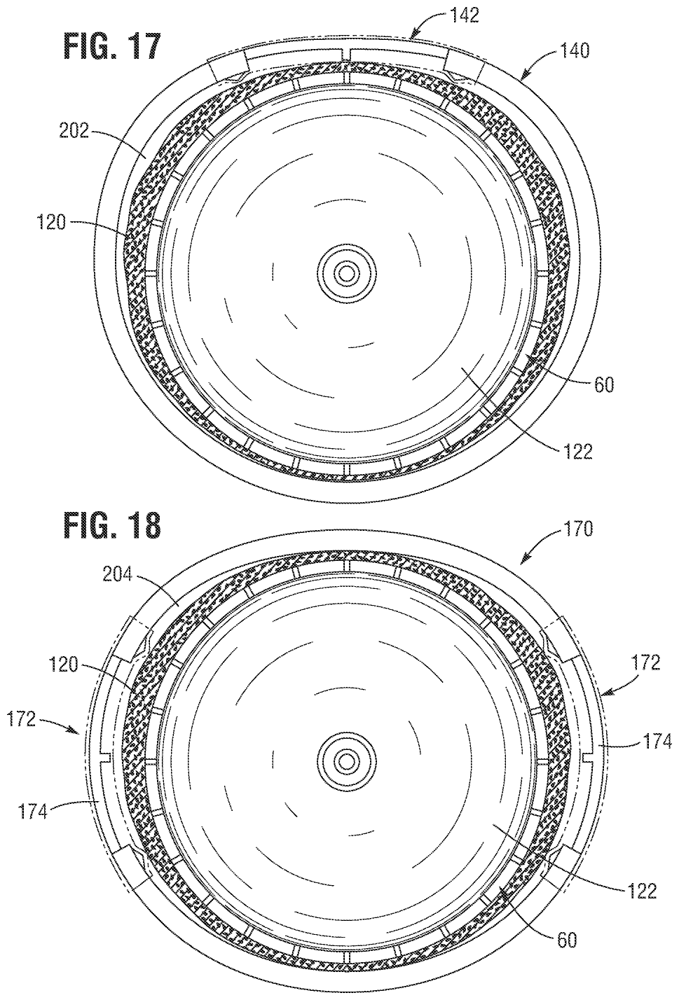

FIG. 17 is a plan view of the expanded balloon 122 and prosthetic heart valve 60 imposed within a mitral annuloplasty ring having the ring core 140 of FIG. 13. The initially D-shaped ring core 140 has the expansion joint 142 on the anterior side, and thus tends to expand to a more circular shape. Moreover, the sealing sleeve 120 compresses unevenly and migrates into the crescent-shaped gaps 202 that remain between the two structures. Depending on the size of the gaps 202 and configuration of the sealing sleeve 120 most of not all of the gaps may be plugged.

FIG. 18 is a plan view of the expanded balloon 122 and prosthetic heart valve 60 imposed within a mitral annuloplasty ring having the ring core 170 of FIG. 16A. The initially D-shaped ring core 170 has the expansion joints 172 on both lateral sides 174, and thus tends to expand to a more circular shape. The sealing sleeve 120 compresses unevenly and migrates into the crescent-shaped gaps 204 that remain between the two structures. Again, depending on the size of the gaps 204 and configuration of the sealing sleeve 120, most if not all of the gaps may be plugged.

While the foregoing is a complete description of the preferred examples, various alternatives, modifications, and equivalents may be used. Moreover, it will be obvious that certain other modifications may be practiced within the scope of the appended claims.

What is claimed is:

1. An annuloplasty ring for mitral valve remodeling adapted for a valve-in-ring procedure, comprising:
an inner core defining a continuous peripheral shape and a suture-permeable tubular interface surrounding the inner core, the inner core being formed of a semi-rigid remodeling material that will resist distortion when subjected to the physiological forces imparted thereon by the mitral valve annulus of an operating human heart, the peripheral shape being generally a D-shape with a substantially straight anterior side diametrically across from a more rounded posterior side with arcuate lateral sides therebetween, and the peripheral shape is oriented around a central axis at an intersection of a minor axis across the midpoints of the anterior and posterior sides and a major axis across the lateral sides, wherein,
the inner core has radially thick and thin regions along discrete segments around the peripheral shape configured to enable different rates of bending when an internal dilatory force is introduced within the annuloplasty ring and the thick and thin regions are positioned to urge the D-shaped peripheral shape to a more circular shape when expanded.

2. The annuloplasty ring of claim 1, wherein the inner core has a first radially thick region at least along the posterior side relative to adjacent lateral sides.

3. The annuloplasty ring of claim 2, wherein the first radially thick region is about twice the radial thickness of the lateral sides.

4. The annuloplasty ring of claim 2, wherein the inner core has a second radially thick region along the anterior side relative to adjacent lateral sides.

5. The annuloplasty ring of claim 4, wherein the first and second radially thick regions are about twice the radial thickness of the lateral sides.

6. The annuloplasty ring of claim 4, wherein the second radially thick region has a plurality of spaced radially-outwardly opening notches formed therein.

7. The annuloplasty ring of claim 1, wherein the peripheral shape is asymmetric across the minor axis.

8. The annuloplasty ring of claim 7, wherein the peripheral shape is three-dimensional and bows down on one side of the minor axis from the anterior side.

9. The annuloplasty ring of claim 1, wherein the inner core is formed with a solid cross-section and made of a semi-rigid metal.

10. The annuloplasty ring of claim 9, wherein the semi-rigid metal is selected from the group consisting of nitinol, cobalt-chromium, stainless steel and a titanium alloy.

11. The annuloplasty ring of claim 1, further including at least one expansion joint around the peripheral shape along the inner core which enables a circumference of the inner core to expand when subjected to the internal dilatory force.

12. An annuloplasty ring for mitral valve remodeling adapted for a valve-in-ring procedure, comprising:
an inner core defining a continuous peripheral shape and a suture-permeable tubular interface surrounding the inner core, the inner core being formed of a semi-rigid remodeling material that will resist distortion when subjected to the physiological forces imparted thereon by the mitral valve annulus of an operating human heart, the peripheral shape being generally a D-shape with a substantially straight anterior side diametrically across from a more rounded posterior side with arcuate lateral sides therebetween, and the peripheral shape is oriented around a central axis at an intersection of a minor axis across the midpoints of the anterior and posterior sides and a major axis across the lateral sides, wherein,
the inner core has radial slits opening to both inner and outer edges around the peripheral shape configured to enable different rates of bending along discrete segments of the inner core when an internal dilatory force is introduced within the annuloplasty ring and the radial slits are positioned to urge the D-shaped peripheral shape to a more circular shape when expanded.

13. The annuloplasty ring of claim 12, wherein there are a first plurality of the radial slits opening outwardly and spaced along the anterior side.

14. The annuloplasty ring of claim 13, wherein there are a second plurality of the radial slits opening outwardly and spaced along the posterior side.

15. The annuloplasty ring of claim 12, wherein there are a plurality of the radial slits opening inwardly and spaced along the lateral sides.

16. The annuloplasty ring of claim 12, wherein each of the radial slits is formed by a substantially straight channel extending radially from the respective edge of the inner core to an enlarged closed relief area.

17. The annuloplasty ring of claim 16, wherein each of the closed relief areas are circular.

18. The annuloplasty ring of claim 12, wherein the peripheral shape is asymmetric across the minor axis.

19. The annuloplasty ring of claim 18, wherein the peripheral shape is three-dimensional and bows down on one side of the minor axis from the anterior side.

20. The annuloplasty ring of claim 12, wherein the inner core is formed with a solid cross-section and made of a semi-rigid metal.

21. The annuloplasty ring of claim 20, wherein the semi-rigid metal is selected from the group consisting of nitinol, cobalt-chromium, stainless steel and a titanium alloy.

22. The annuloplasty ring of claim 12, further including at least one expansion joint around the peripheral shape along the inner core which enables a circumference of the inner core to expand when subjected to the internal dilatory force.

23. A combination of an annuloplasty ring for mitral valve remodeling adapted for a valve-in-ring procedure and an expandable prosthetic heart valve, comprising:
an annuloplasty ring having an inner core defining a continuous peripheral shape and a suture-permeable tubular interface surrounding the inner core, the inner core being formed of a semi-rigid remodeling material that will resist distortion when subjected to the physiological forces imparted thereon by the mitral valve annulus of an operating human heart, the peripheral shape being generally a D-shape with a substantially straight anterior side diametrically across from a more rounded posterior side with arcuate lateral sides therebetween, and the peripheral shape is oriented around a central axis at an intersection of a minor axis across the midpoints of the anterior and posterior sides and a major axis across the lateral sides, wherein, the inner core is configured to expand from the D-shaped peripheral shape to a more circular shape when expanded by an internal dilatory force; and
an expandable prosthetic heart valve having an expandable support frame made to plastically expand with a generally circular cross-section when subjected to an outward dilatory force, the support frame being covered by a fabric cover surrounded by an attached sealing sleeve formed of a compressible or inelastic material, wherein the heart valve has a contracted shape for advancement within the annuloplasty ring and an expanded shape sized to contact and expand the annuloplasty ring and convert the peripheral shape to a more circular shape, wherein the sealing sleeve expands into gaps between the heart valve and annuloplasty ring and is adapted to form radially thicker and thinner regions around a circumference of the heart valve.

24. The combination of claim 23, wherein the inner core has radially thick and thin regions along discrete segments around the peripheral shape configured to enable different rates of bending when the internal dilatory force is introduced within the annuloplasty ring.

25. The combination of claim 23, wherein the inner core has radial slits opening to both inner and outer edges around the peripheral shape configured to enable different rates of bending along discrete segments of the inner core when the internal dilatory force is introduced within the annuloplasty ring.

26. The combination of claim 23, further including at least one expansion joint around the peripheral shape of the inner core which enables a circumference of the inner core to expand when subjected to the internal dilatory force.

27. The combination of claim 23, wherein the sealing sleeve attaches to the fabric cover along one circumferential line so as to decouple axial foreshortening of the expandable support frame from the sealing sleeve.

28. The combination of claim 23, wherein the sealing sleeve comprises an annular bladder having inner and outer walls holding a fluid therein.

29. The combination of claim 28, wherein the annular bladder is made of polyethylene terephthalate and the fluid is silicone.

30. The combination of claim 23, wherein the sealing sleeve comprises a solid material which is plastically deformable.
